(19) United States
(12) Patent Application Publication
(10) Pub. No.: US 2026/0191748 A1
(43) Pub. Date: Jul. 9, 2026

(54) PROCESS FOR TREATING HUMAN PERSPIRATION AND ASSOCIATED COMPOSITION

(71) Applicant: L'OREAL, Paris (FR)

(72) Inventors: Caroline MENA, Chevilly Larue (FR); Charlotte GOZLAN, Chevilly Larue (FR); Widad AJILI, Chevilly Larue (FR)

(73) Assignee: L'OREAL, Paris (FR)

(21) Appl. No.: 19/133,279

(22) PCT Filed: Nov. 28, 2023
(86) PCT No.: PCT/EP2023/083382

§ 371 Date: May 28, 2025

(30) Foreign Application Priority Data
Nov. 30, 2022 (FR) 2212560

(51) Int. Cl. A61K 8/20; A61K 8/24; A61K 8/365; A61K 8/368; A61K 8/73; A61Q 15/00

(52) U.S. Cl. CPC A61K 8/20; A61K 8/24; A61K 8/365; A61K 8/368; A61K 8/73; A61Q 15/00; A61K 2800/30; A61K 2800/882; A61K 2800/884

(57) ABSTRACT

The present invention relates to a composition comprising, in a cosmetically acceptable aqueous medium, magnesium chloride, dipotassium phosphate, xanthan gum, capryloyl salicylic acid, and at least one modulating agent chosen from among the mono- or poly-carboxylic acids, optionally hydroxylated, in free or salified form. It also relates to the associated method.

This invention relates to a cosmetic method for the treatment of human perspiration and optionally body odors resulting from the perspiration, as well as an associated cosmetic composition.

The underarms as well as certain other parts of the body are generally the place of several discomforts that can stem directly or not from perspiration problems. These phenomena often cause unpleasant and uncomfortable sensations which are primarily due to the presence of sweat resulting from perspiration that can, in certain cases, make the skin moist and wet clothing, in particular at the underarms or the back, as such leaving visible traces. Moreover, the presence of sweat can generate the discharging of body odors that are most of the time unpleasant. Such discomforts are to be taken into account, including in the case of moderate perspiration.

In the cosmetics field, it is as such well known to use a topical application of deodorant products containing substances that have the effect of masking, absorbing, improving and/or reducing the unpleasant odor resulting from the decomposition of human sweat and remedying the problems mentioned hereinabove. These products are in general available in the form of roll-on, sticks, aerosol or spray.

There are also antiperspirant compositions on the market that generally include aluminum and/or zirconium hydrochlorides. These substances make it possible to reduce the sweat flow by forming a plug on the sweat duct.

However, the use of these substances at high concentrations in order to obtain good effectiveness causes difficulties in terms of the formulation. In addition, it has been observed that the antiperspirant effectiveness of these substances is limited when they are used alone.

In order to overcome all of these disadvantages mentioned hereinabove, it has been proposed to seek other effective active substances, that are tolerated by the skin and easy to formulate.

The approaches recently worked on include the use of magnesium oxide (MgO) in various dosage forms: this active ingredient has proven its effectiveness as a deodorant, but the formula needs to be improved.

Another approach is that described in WO2019/072831: this application describes a cosmetic method for the treatment of human perspiration and optionally body odors resulting from perspiration, which includes the use of at least one cation Xⁿ⁺ of valency n, of at least one anion Y^{m-} of valency m, and of at least one modulating agent. However, these described cations and anions are not equivalent and the dosage forms need to be improved.

There is therefore a real need to implement on the skin an agent intended for the treatment of human perspiration that does not have all of the disadvantages described hereinabove, i.e. that provides a satisfactory antiperspirant effect and that is well tolerated by the skin. Furthermore, there is a need for such a deodorant agent for the treatment of human perspiration that is easy to formulate, non-irritating, and stable during storage.

The applicant discovered surprisingly that this objective could be reached with an aqueous composition comprising magnesium chloride (MgCl₂), dipotassium phosphate (K₂HPO₄), xanthan gum, and capryloyl salicylic acid. The resulting composition is easy to formulate and stable, and is effective as a deodorant.

This invention therefore relates to a composition, in a cosmetically acceptable aqueous medium, magnesium chloride (MgCl₂), dipotassium phosphate (K₂HPO₄), xanthan gum, capryloyl salicylic acid, and at least one modulating agent chosen from among the mono- or poly-carboxylic acids, optionally hydroxylated, in free or salified form.

The term “cosmetically acceptable aqueous medium” denotes an aqueous medium compatible with the skin and/or appendages or mucosa thereof, having a pleasant color, odor and texture and not giving rise to unacceptable discomfort (such as tightness), liable to dissuade the consumer from using the composition.

Said cosmetically acceptable medium is also a medium that does not leave any traces during the application thereof, and that thus does not stain clothing.

Said cosmetically acceptable medium is aqueous, i.e. it comprises an aqueous phase.

This invention also relates to a cosmetic method for treating human perspiration and optionally body odors resulting from perspiration (referred to hereinafter as the “method according to the invention”), comprising the application of a composition according to the invention to skin.

Lastly, this invention relates to a cosmetic method for the treatment of human perspiration and optionally body odors from perspiration (hereinafter “method 2 according to the invention”), which includes:
(i) either the mixing just before use of at least one composition A and of at least one composition B, said compositions A and B being packaged separately, followed by the application of the resulting mixture on the surface of the skin;
(ii) or the application on the surface of the skin simultaneously or sequentially of at least one composition A and of at least one composition B packaged separately;
(iii) or the application on the surface of the skin of a composition comprising in the same support at least one composition A and of at least one composition B;
   said composition A comprising in a cosmetically acceptable aqueous medium at least magnesium chloride (MgCl₂);
   said composition B comprising in a cosmetically acceptable aqueous medium at least dipotassium phosphate (K₂HPO₄);
   said composition A and/or said composition B comprising xanthan gum;
   said composition A and/or said composition B comprising capryloyl salicylic acid; and
   said composition A and/or said composition B comprising also comprising at least one modulating agent chosen from among the mono- or poly-carboxylic acids, potentially hydroxylated, in free or salified form.

“Sequential” means a successive administration.

“Same holder” means that compositions A and B according to the invention are present in the same packaging, in particular in a two-compartment packaging that allows for the simultaneous application of compositions A and B.

### Composition

The composition according to the invention comprises, in a cosmetically acceptable aqueous medium, magnesium chloride (MgCl₂), dipotassium phosphate (K₂HPO₄), xanthan gum, capryloyl salicylic acid, and at least one modulating agent chosen from among the mono- or poly-carboxylic acids, potentially hydroxylated, in free or salified form.

### Aqueous Phase

The composition comprises an aqueous phase (also referred to as a cosmetically acceptable aqueous medium). In particular, it is formulated in an aqueous gel.

The aqueous phase of the composition contains water and generally other solvents soluble in water or miscible with water. The water-soluble or miscible solvents comprise short-chain mono-alcohols, for example at C₁-C₄, such as ethanol, isopropyl alcohol; diols or polyols such as ethyleneglycol, 1,2-propyleneglycol, 1,3-butylene glycol, hexyleneglycol, diethyleneglycol, dipropylene glycol, 2-ethoxyethanol, diethylene glycol monomethylether, triethylene glycol monomethylether and sorbitol, preferably 1,2-propyleneglycol.

Preferably, the composition comprises from 1% to 30% by weight of water-soluble or water-miscible solvent(s) in water with respect to the total weight of the composition, preferably from 2% to 25% by weight, preferably from 4% to 22% by weight.

Preferably, the composition comprises at least 50% by weight of water with respect to the total weight of the composition, preferably at least 55% by weight, preferably at least 70% by weight. Preferably, the composition comprises from 50% to 90% by weight of water with respect to the total weight of the composition, preferably from 55% to 85% by weight, preferably from 55% to 80% by weight.

### Magnesium Chloride (MgCl₂)

Magnesium chloride (MgCl₂) is a water-soluble salt. In terms of this invention, the term “water-soluble salt” means any salt that, after having been completely dissolved with stirring at 0.5% in a solution of water at a temperature of 25° C., leads to a solution comprising a quantity of insoluble salt less than 0.05% by weight. In particular, magnesium chloride (MgCl₂) comprises the cation Mg²⁺.

Preferably, the composition according to the invention comprises an aqueous phase and preferably has a pH between 2 and 6, preferably between 4 and 6. If necessary, the pH is adjusted with a cosmetically acceptable organic or mineral acid or base. Such an acid or such a base are those conventionally used in cosmetics.

Preferably, the composition comprises magnesium chloride at a concentration of between 1% and 30% by weight with respect to the total weight of the composition, preferably between 3% and 25% by weight, preferably between 5% and 20% by weight.

Dipotassium Phosphate (K₂HPO₄)

The composition according to the invention also comprises dipotassium phosphate (K₂HPO₄). This dipotassium phosphate (K₂HPO₄) is also a water-soluble salt. In particular, dipotassium phosphate (K₂HPO₄) comprises the anion HPO₄²⁻.

Preferably, the composition comprises dipotassium phosphate (K₂HPO₄) at a concentration of between 1% and 25% by weight with respect to the total weight of the composition, preferably between 3% and 20% by weight, preferably between 5% and 15% by weight.

The weight ratio of the magnesium chloride to the dipotassium phosphate preferably varies from 3:1 to 1:1, preferably from 2:1 to 1:1, and more preferably from 1.5:1 to 1:1.

The total concentration of and magnesium chloride and dipotassium phosphate preferably varies from 7% to 40% by weight, preferably between 10% and 30% by weight, and more preferably between 15% and 25% by weight in relation to the total weight of the composition.

### Modulating Agent

The composition according to the invention also comprises comprising at least one modulating agent chosen from among the mono- or poly-carboxylic acids, potentially hydroxylated, in free or salified form.

Preferably, the polycarboxylic acids are chosen from among the di- and tri-carboxylic acids, optionally hydroxylated. Such hydroxylated carboxylic acids are also referred to as hydroxy acids.

Preferably, the modulating agent is chosen from propionic acid, citric acid, tartaric acid, lactic acid, malic acid, succinic acid, glutaric acid, itaconic acid, and mixtures thereof, in free or salified form.

Preferably, the modulating agent is chosen from the tricarboxylic acids, optionally hydroxylated, preferably from the hydoxylated tricarboxylic acids.

The optionally hydroxylated mono- or poly-carboxylic acids are present in the composition in free or salified form.

When present in salified form, they may be present in the form of an alkaline metal salt or an alkaline-earth metal salt, preferably in the form of a sodium or potassium salt.

Preferably, the modulating agent is a mixture of an optionally hydroxylated tricarboxylic acid in free form and an optionally hydroxylated tricarboxylic acid in salified form.

Preferably, the modulating agent is a mixture of a hydroxylated tricarboxylic acid in free form and a hydroxylated tricarboxylic acid in salified form.

Preferably, the modulating agent is a mixture of citric acid and sodium citrate.

Preferably, the modulating agent is present in the composition at a concentration of between 0.1% and 10% by weight with respect to the total weight of the composition, preferably between 0.5% and 5% by weight, preferably between 1% and 4% by weight.

### Xanthan Gum

The composition according to the invention comprises xanthan gum.

Preferably, the xanthan gum is present in the composition at a concentration of between 0.1% and 7% by weight with respect to the total weight of the composition, preferably between 0.5% and 5% by weight, preferably between 0.8% and 2% by weight.

### Deodorant Agent

The composition according to the invention comprises capryloyl salicylic acid (also referred to as 2-hydroxy-5-(1-oxooctyl)benzoic acid).

This compound is a deodorant agent. The term “deodorant agent” denotes in the context of the present invention any agent, alone, with the effect of masking, absorbing, enhancing and/or reducing the unpleasant odor resulting from the decomposition of human sweat.

Preferably, capryloyl salicylic acid is present in the composition at a concentration of between 0.05% and 5% by weight in relation to the total weight of the composition, preferably between 0.08% and 3% by weight, preferably between 0.1% and 1% by weight.

### Preservatives and Other Additives

The composition according to the invention may comprise at least one preservative and/or at least one additive.

As preservatives, let us mention in particular phenoxyethanol, caprylyl glycol, benzyl alcohol, and mixtures thereof.

Preferably, the composition according to the invention is substantially free of caprylyl glycol. Substantially free of caprylyl glycol means that the composition comprises less than 0.3% by weight, preferably less than 0.1% by weight of caprylyl glycol with respect to the total weight of the composition. Preferably, the composition according to the invention is totally free of caprylyl glycol.

Preferably, the composition according to the invention comprises at least some phenoxyethanol.

The additive may be any customary additive in the cosmetics field, such as a scent or a filler.

Preferably, the composition according invention comprises the following by weight with respect to the total weight of the composition, in a cosmetically acceptable aqueous medium:
at least 55% by weight of water;
magnesium chloride (MgCl₂) at a concentration of between 1% and 30% by weight with respect to the total weight of the composition, preferably between 3% and 25% by weight, preferably between 5% and 20% by weight;
dipotassium phosphate (K₂HPO₄) at a concentration of between 5% and 20%, preferably between 6% and 15% by weight, preferably between 7% and 12% by weight;
Between 0.05% and 5% by weight capryloyl salicylic acid, preferably between 0.08% and 3% by weight, preferably between 0.1% and 1% by weight;
possibly at least one preservative, preferably phenoxyethanol;
between 0.1% and 5% by weight citric acid, preferably between 0.5% and 4% by weight, preferably between 1% and 3% by weight;
between 0.05% and 5% by weight sodium citrate, preferably between 0.1% and 3% by weight, preferably between 0.5% and 2% by weight; and
between 0.5% and 4% by weight xanthan gum, preferably between 0.6% and 2% by weight, preferably between 0.7% and 1.5% by weight.

### Method of Application

This invention also relates to a cosmetic method for treating human perspiration and optionally body odors resulting from perspiration (referred to hereinafter as the “method according to the invention”), comprising the application of a composition according to the invention to skin.

Lastly, this invention relates to a cosmetic method for the treatment of human perspiration and optionally body odors from perspiration (hereinafter “method 2 according to the invention”), which includes:
(i) either the mixing just before use of at least one composition A and of at least one composition B, said compositions A and B being packaged separately, followed by the application of the resulting mixture on the surface of the skin;
(ii) or the application on the surface of the skin simultaneously or sequentially of at least one composition A and of at least one composition B packaged separately;
(iii) or the application on the surface of the skin of a composition comprising in the same support at least one composition A and of at least one composition B;
   said composition A comprising in a cosmetically acceptable aqueous medium at least magnesium chloride (MgCl₂);
   said composition B comprising in a cosmetically acceptable aqueous medium at least dipotassium phosphate (K₂HPO₄);
   said composition A and/or said composition B comprising xanthan gum;
   said composition A and/or said composition B comprising capryloyl salicylic acid; and
   said composition A and/or said composition B comprising also comprising at least one modulating agent chosen from among the mono- or poly-carboxylic acids, potentially hydroxylated, in free or salified form.

In order to obtain an antiperspirant effect on the skin, according to a first alternative embodiment of method 2 according to the invention (embodiment (i)), composition A and composition B are packaged separately and are mixed right before use (extemporaneous mixture), then the resulting mixture is applied to the surface of the skin to be treated.

According to a second alternative embodiment of method 2 according to the invention (embodiment (ii)), composition A and composition B are packaged separately and are applied simultaneously or sequentially to the surface of the skin to be treated. According to this alternative embodiment, when compositions A and B are applied sequentially, the time interval separating the application of composition A from application B can vary from 1 second to 24 hours, more preferably from 10 seconds to 24 hours and even more preferably from 1 minute to 1 hour.

According to a third alternative embodiment of method 2 according to the invention (embodiment (iii)), a composition comprising in the same medium composition A and composition B is applied directly to the surface of the skin. In this embodiment (iii), compositions A and B can be, for example, packaged in a device comprising at least two compartments containing composition A and composition B, respectively, such as a twin-tube, a pump bottle with two compartments, an aerosol device comprising two compartments that can include one or more outlet orifices (mono-nozzle or double-nozzle), a device provided with a perforated wall such as a grid comprising two compartments; a device comprising two compartments each provided with a ball applicator (multi-ball roll-on); a double-stick.

Preferably, compositions A and B are applied sequentially and even more preferably, composition A (comprising at least magnesium chloride) is applied first.

Preferably, the invention also relates to a method for the cosmetic treatment of human perspiration, and optionally body odors associated with human perspiration, comprising the application of an effective quantity of composition A and an effective quantity of composition B to the surface of the skin, particularly the surface of the underarms.

The application time of composition A and/or B to the surface of the skin can vary from 0.5 to 10 seconds, preferably from 1 to 5 seconds. Compositions A and B can be applied several times to the surface of the skin. They can be applied several times, over one day or over several days.

Preferably, the invention also relates to a method for the cosmetic treatment of human perspiration, and optionally body odors associated with human perspiration, comprising a first step for applying composition A to the surface of the skin, particularly the surface of the underarms, and then a second step for applying composition B to the same surface.

### Dosage Forms

The composition according to the invention may be in any of the dosage forms conventionally used for topical application and particularly in the form of aqueous or hydroalcoholic gels, or in the form of aqueous sticks (i.e. solid).

It is preferably in the form of an aqueous gel or aqueous stick (i.e. solid).

It may also, by the addition of an oily or oil phase, be in the form of dispersions such as lotion, emulsions of liquid or semi-liquid consistency such as milk, obtained by dispersing an oily phase in an aqueous phase (O/W) or conversely (W/O), or suspensions or emulsions of soft, semi-solid or solid consistency such as cream or gel, or multiple emulsions (W/O/W or O/W/O), microemulsions, ionic and/or non-ionic type vesicle dispersions, or wax/aqueous phase dispersions. These compositions are prepared using routine methods.

The composition may be particularly packaged in pressurized form in an aerosol device or in a pump bottle; packaged in a device equipped with a perforated wall particularly a grid; packaged in a device equipped with a roll-on applicator; packaged in stick form, in loose or compact powder form. In this respect, it contains the ingredients generally used in this type of products and well-known to a person skilled in the art.

According to another particular embodiment of the invention, the composition may be solid, particularly in the form of a stick; in the form of a loose or compact powder.

“Solid composition” means that the measurement of the maximum force measured by means of texturometric analysis on inserting a probe in the sample of formula should be at least equal to 0.25 Newton, in particular at least equal to 0.30 Newton, particularly at least equal to 0.35 Newton, assessed under precise measurement conditions as follows: the formulas are poured hot into jars 4 cm in diameter and 3 cm high. Cooling is performed at ambient temperature. The hardness of the formulas produced is measured after waiting 24 hours. The jars containing the samples are characterized by means of texturometric analysis using a texturometer such as that marketed by Rheo TA-XT2, according to the following protocol: a 5 mm diameter stainless steel ball type probe is brought into contact with the sample at a speed of 1 mm/s. The measurement system detects the interface with the sample with a detection threshold equal to 0.005 Newton. The probe is inserted 0.3 mm into the sample, at a rate of 0.1 mm/s. The measurement apparatus records the progression of the compression force measured overtime, during the penetration phase. The hardness of the sample corresponds to the mean of the maximum values of the force detected during penetration, for at least 3 measurements.

The composition can also comprise at least one deodorant agent and/or one additional antiperspirant agent.

By way of illustration of these additional deodorant agents, mention can be made in particular of bacteriostatic agents or bactericidal agents acting on germs of underarm odor, such as 2,4,4′-trichloro-2′-hydroxydiphenylether (Triclosan), 2,4-dichloro-2′-hydroxydiphenylether, 3′,4′,5′-trichlorosalicylanilide, 1-(3′,4′-dichlorophenyl)-3-(4′-chlorophenyl)urea (Triclocarban) or 3,7,11-trimethyldodeca-2,5,10-trienol (Farnesol); quaternary ammonium salts such as cetyltrimethylammonium salts, cetylpyridinium salts; polyols such as those of the glycerin type, 1,3-propanediol (ZEMEA PROPANEDIOL® sold by Dupont Tate and Lyle Bioproducts), 1,2-decanediol (Symclariol® from Symrise); glycerin derivatives such as for example Caprylic/Capric Glycerides (CAPMUL MCM® from Abitec), Caprylate or glyceryl caprylate (DERMOSOFT GMCY® and DERMOSOFT GMC® respectively from STRAETMANS), Polyglyceryl-2 Caprate (DERMOSOFT DGMC® from STRAETMANS), biguanide derivatives such as polyhexamethylene biguanide salts; chlorhexidine and salts thereof; 4-Phenyl-4,4-dimethyl-2-butanol (SYMDEO MPP® from Symrise); cyclodextrins; or alum.

The additional deodorant agents can be present preferably in the compositions according to the invention in mass concentrations ranging from 0.01% to 10% by weight relative to the total weight of the composition.

By way of illustration of additional antiperspirant agents, mention can be made in particular of aluminum and/or zirconium antiperspirant salts or complexes, preferably chosen from aluminum halohydrates; aluminum and zirconium halohydrates, zirconium hydroxychloride and aluminum hydroxychloride complexes with or without an amino acid such as those described in U.S. Pat. No. 3,792,068.

Among the aluminum salts, mention can be made in particular of aluminum chlorohydrate in activated or non-activated form, aluminum chlorohydrex, aluminum chlorohydrex polyethyleneglycol complex, aluminum chlorohydrex propyleneglycol complex, aluminum dichlorohydrate, aluminum dichlorohydrex polyethyleneglycol complex, aluminum dichlorohydrex propyleneglycol complex, aluminum sesquichlorohydrate, aluminum sesquichlorohydrex polyethylene glycol complex, aluminum sesquichlorohydrex propyleneglycol complex, aluminum sulfate buffered by aluminum and sodium lactate.

Among the aluminum and zirconium salts, mention can be made in particular of aluminum zirconium octachlorohydrate, aluminum zirconium pentachlorohydrate, aluminum zirconium tetrachlorohydrate, aluminum zirconium trichlorohydrate.

Zirconium hydroxychloride and aluminum hydroxychloride complexes with an amino acid are generally known under the name ZAG (when the amino acid is glycine). Among these products, mention can be made of complexes of aluminum zirconium octachlorohydrex glycine, aluminum zirconium pentachlorohydrex glycine, aluminum zirconium tetrachlorohydrex glycine and aluminum zirconium trichlorohydrex glycine.

Aluminum sesquichlorohydrate is in particular sold under the trade name REACH 301® by SUMMITREHEIS.

Among the aluminum and zirconium salts, mention can be made of the complexes of zirconium hydroxychloride and aluminum hydroxychloride with an amino acid such as glycine having for INCI name: ALUMINUM ZIRCONIUM TETRACHLOROHYDREX GLY for example that sold under the trade name REACH AZP-908-SUF® by SUMMITREHEIS.

Use will be made more particularly of aluminum chlorohydrate in activated form or not sold under the trade names LOCRON S FLA®, LOCRON P, LOCRON L.ZA by CLARIANT; under the trade names MICRODRY ALUMINUM CHLOROHYDRATE®, MICRO-DRY 323®, CHLORHYDROL 50, REACH 103, REACH 501 by SUMMITREHEIS; under the trade name WESTCHLOR 200® by WESTWOOD; under the trade name ALOXICOLL PF 40® by GUILINI CHEMIE; CLURON 50%® by INDUSTRIA QUIMICA DEL CENTRO; CHLOROHIDROXIDO ALUMINIO SO A 50%® by FINQUIMICA. As another antiperspirant agent, mention can be made of the particles of expanded perlite such as those obtained by the method of expansion described in the U.S. Pat. No. 5,002,698.

Preferably, the composition comprises less than 5% by weight of aluminum salt, preferably less than 3% by weight, preferably less than 1% by weight. Preferably, it is totally free of aluminum salt.

Throughout the description, including the claims, the expression “comprising a” must be understood as being synonymous with “comprising at least one”, unless mentioned otherwise.

The expressions “between . . . and . . . ” and “ranging from . . . to . . . ” are to be understood to be inclusive of the limits, unless specified otherwise.

In the description and the examples, unless mentioned otherwise, the percentages are weight percentages. The percentages are therefore expressed by weight in relation to the total weight of the composition (% w/w). The temperature is expressed in degrees Celsius unless mentioned otherwise, and the pressure is the atmospheric pressure, unless mentioned otherwise.

The invention is illustrated in more detail by the non-limiting examples presented hereinafter.

### EXAMPLES

### Example 1: Compositions

The gel formulas below have been prepared by mixing in water:

Formulas C1 an C2 are according to the invention, whereas formula CC1 is for comparison (no capryloyl salicylic acid).

TABLE 1

| | CC1 | C1 | C2 |
|---|---|---|---|
| Ingredient | (% w/w) | (% w/w) | (% w/w) |
| Xanthan gum | 0.9 | 0.9 | 0.9 |
| CAPRYLYL GLYCOL | 0.5 | 0.5 | |
| PROPYLENE GLYCOL | | 5 | 5 |
| Citric acid | 1.5 | 1.5 | 1.5 |
| SODIUM CITRATE | 0.7 | 0.7 | 0.7 |
| Water | QS 100 | QS 100 | QS 100 |
| Dipotassium phosphate | 8.6 | 8.6 | 8.6 |
| (K₂HPO₄) | | | |
| Magnesium chloride (MgCl₂) | 10 | 10 | 10 |
| Capryloyl salicylic acid | | 0.2 | 0.2 |
| Perfume | | | 1.2 |
| Phenoxyethanol | | | 0.5 |

### Example 2: Evaluating Antibacterial Performance

The evaluation of the antimicrobial properties of formulas CC1, C1, and C2 in example 1 is done using the following protocol:

Summarizing, the biocidal effect of the formulas is evaluated on strains of interest after 2, 6, and 24 hours of contact versus a placebo (i.e. Medium without formula), in an adapted culture medium. The logarithmic reduction of the number of viable organisms is measured at each contact time.

The model strain for “underarm odor” is:
Corynebacterium freneyi CIP 52.16 (ex Corynebacterium xerosis)

This strain is brought into contact with the formula being tested in a culture medium, in the following proportions:
10% of the inoculum at 10⁸ germs/ml,
10% of the formula being tested, and
80% culture medium (Trypcase Soy broth).

At the same time, a growth control, in which the formula is replaced with a diluent, is prepared under the same conditions.

The samples are placed in an incubator at a temperature of 35° C. (optimum growth temperature for the strain) with continuous stirring during the test.

After 2, 6, and 24 hours of contact, the number of viable microorganisms remaining in the mixture is evaluated.

The results are expressed as a logarithm of the number of micro-organisms per milliliter of mixture.

The result obtained with the formula being tested is compared to the results of the growth control, and the difference is expressed as a Log number difference at T equals 24 hours.

The count (Log CFU/ml) for the control at 24 hours is greater than 8 (about 8.5).

The count (Log CFU/ml) for CC1 at 24 hours is greater than 8 (about 8.3).

The count (Log CFU/ml) for C1 at 24 hours is about 3.3.

The count (Log CFU/ml) for C2 as of 2 hours is about 2.3 (=detection threshold).

By comparing formulas CC1 and C1, the antimicrobial activity of C1 is found to be significantly improved by adding capryloyl salicylic acid (−4 Log at 24 hours).

With formula C2 according to the invention, all the bacteria are killed after 2 hours.

Formulas C1 and C2 according to the invention thus demonstrate very strong antimicrobial activity.

### Example 3: Compositions

The gel formulas below have been prepared by mixing in water:

Formulas C3 to C5 are according to the invention, whereas formulas CC2 to CC4 are for comparison (no capryloyl salicylic acid).

TABLE 2

| | CC2 | C3 |
|---|---|---|
| Ingredient | (% w/w) | (% w/w) |
| Xanthan gum | 0.9 | 0.9 |
| PROPYLENE GLYCOL | | 20 |
| Citric acid | 1.5 | 1.5 |
| SODIUM CITRATE | 0.7 | 0.7 |
| Water | QS 100 | QS 100 |
| Dipotassium phosphate | 8.6 | 8.6 |
| (K₂HPO₄) | | |
| Magnesium chloride (MgCl₂) | 10 | 10 |
| Capryloyl salicylic acid | | 0.2 |
| Phenoxyethanol | 0.5 | 0.5 |

TABLE 3

| | CC3 | C4 |
|---|---|---|
| Ingredient | (% w/w) | (% w/w) |
| Xanthan gum | 0.9 | 0.9 |
| Citric acid | 1.5 | 1.5 |
| SODIUM CITRATE | 0.7 | 0.7 |
| Water | QS 100 | QS 100 |
| Dipotassium phosphate | 8.6 | 8.6 |
| (K₂HPO₄) | | |
| Magnesium chloride (MgCl₂) | 10 | 10 |
| Capryloyl salicylic acid | | 0.2 |
| Perfume | 1.2 | 1.2 |
| Phenoxyethanol | 0.5 | 0.5 |

TABLE 4

| | CC4 | C5 |
|---|---|---|
| Ingredient | (% w/w) | (% w/w) |
| Xanthan gum | 0.9 | 0.9 |
| CAPRYLYL GLYCOL | | 0.5 |
| Citric acid | 1.5 | 1.5 |
| SODIUM CITRATE | 0.7 | 0.7 |
| Water | QS 100 | QS 100 |
| Dipotassium phosphate | 8.6 | 8.6 |
| (K₂HPO₄) | | |
| Magnesium chloride (MgCl₂) | 10 | 10 |
| Capryloyl salicylic acid | | 0.2 |
| Perfume | 1.2 | 1.2 |
| Phenoxyethanol | 0.5 | |

### Example 4: Evaluating Deodorant Performance

The deodorant properties of formulas CC2-CC4 and C3-C5 in example 3 are evaluated using the following protocol:

### Methodology

Panel: 10 panelists trained for objective evaluation. Performance checked for repeatability, discrimination, and homogeneity

Volunteers: 18 volunteers with strong underarm odor

Application: 7 days of multi-application (4) washing Random product application

Quantity applied each day: 0.4 g±0.05 roll-on & stick

1.2 g±0.05 aerosol

Evaluation: intensity of the bad smell, description of the bad smell and intensity of the scent.

At T0, T4, and T24 hours after the 4th application. Sequential monadic.

The results show the following values:

TABLE 5

| | CC2 | C3 |
|---|---|---|
| Value | (% w/w) | (% w/w) |
| T0 | −30 | −35 |
| T4 h | −29 | −38 |
| T24 h | −25 | −33 |

Formula C3 according to the invention is significantly more effective than comparison formula CC2 after 4 and 24 hours.

TABLE 6

| | CC3 | C4 |
|---|---|---|
| Value | (% w/w) | (% w/w) |
| T0 | −40 | −40 |
| T4 h | −35 | −41 |
| T24 h | −32 | −35 |

Formula C4 according to the invention is slightly more effective than comparison formula CC3 at T0 and 4 hours, but comparable at 24 hours.

TABLE 7

| | CC4 | C5 |
|---|---|---|
| Value | (% w/w) | (% w/w) |
| T0 | −40 | −40 |
| T4 h | −32 | −39 |
| T24 h | −20 | −30 |

Formula C5 according to the invention is more effective than comparison formula CC4 after 4 and 24 hours.

1. A composition comprising, in a cosmetically acceptable aqueous medium, magnesium chloride, dipotassium phosphate, xanthan gum, capryloyl salicylic acid, and at least one modulating agent chosen from among the mono- or poly-carboxylic acids, optionally hydroxylated, in free or salified form.

2. The composition according to claim 1, characterized in that it comprises magnesium chloride at a concentration of between 1% and 30% by weight with respect to the total weight of the composition.

3. A composition according to claim 1, characterized in that it comprises dipotassium phosphate at a concentration of between 1% and 25% by weight with respect to the total weight of the composition.

4. A composition according to claim 1, characterized in that the weight ratio of the magnesium chloride to the dipotassium phosphate varies from 3:1 to 1:1.

5. A composition according to claim 1, characterized in that the modulating agent is chosen from propionic acid, citric acid, tartaric acid, lactic acid, malic acid, succinic acid, glutaric acid, itaconic acid, and mixtures thereof, in free or salified form.

6. A composition according to claim 1, characterized in that the modulating agent is a mixture of a hydroxylated tricarboxylic acid in free form and a hydroxylated tricarboxylic acid in salified form.

7. A composition according to claim 1, characterized in that the modulating agent is a mixture of citric acid and sodium citrate.

8. A composition according to claim 1, characterized in that the modulating agent is present in the composition at a concentration of between 0.1% and 10% by weight with respect to the total weight of the composition.

9. A composition according to claim 1, characterized in that the xanthan gum is present in the composition at a concentration of between 0.1% and 7% by weight with respect to the total weight of the composition.

10. A composition according to claim 1, characterized in that the capryloyl salicylic acid is present in the composition at a concentration of between 0.05% and 5% by weight in relation to the total weight of the composition.

11. A composition according to claim 1, characterized in that it comprises at least 50% by weight of water with respect to the total weight of the composition.

12. A composition according to claim 1, characterized in that it is substantially free of caprylyl glycol.

13. A composition according to claim 1, characterized in that it comprises, in a cosmetically acceptable aqueous medium, the following by weight with respect to the total weight of the composition:
at least 55% by weight of water;
magnesium chloride at a concentration of between 1% and 30% by weight with respect to the total weight of the composition;
dipotassium phosphate at a concentration of between 5% and 20%;
between 0.05% and 5% by weight capryloyl salicylic acid;
possibly at least one preservative;
between 0.1% and 5% by weight citric acid;
between 0.05% and 5% by weight sodium citrate; and
between 0.5% and 4% by weight xanthan gum.

14. A cosmetic method for treating human perspiration and optionally body odors resulting from perspiration, comprising the application of a composition according to claim 1 to skin.

15. A cosmetic method for the treatment of human perspiration and optionally body odors resulting from perspiration, comprising:
(i) either the mixing just before use of at least one composition A and of at least one composition B, said compositions A and B being packaged separately, followed by the application of the resulting mixture on the surface of the skin;
(ii) or the application on the surface of the skin simultaneously or sequentially of at least one composition A and of at least one composition B packaged separately;
(iii) or the application on the surface of the skin of a composition comprising in the same support at least one composition A and of at least one composition B;
said composition A comprising in a cosmetically acceptable aqueous medium at least magnesium chloride;
said composition B comprising in a cosmetically acceptable aqueous medium at least dipotassium phosphate;
said composition A and/or said composition B comprising xanthan gum;
said composition A and/or said composition B comprising capryloyl salicylic acid; and
said composition A and/or said composition B comprising also comprising at least one modulating agent chosen from among the mono- or poly-carboxylic acids, potentially hydroxylated, in free or salified form.

16. A composition according to claim 2, characterized in that it comprises dipotassium phosphate at a concentration of between 1% and 25% by weight with respect to the total weight of the composition.

17. A composition according to claim 2, characterized in that the weight ratio of the magnesium chloride to the dipotassium phosphate varies from 3:1 to 1:1.

18. A composition according to claim 2, characterized in that the weight ratio of the magnesium chloride to the dipotassium phosphate varies from 3:1 to 1:1.

19. A composition according to claim 2, characterized in that the modulating agent is chosen from propionic acid, citric acid, tartaric acid, lactic acid, malic acid, succinic acid, glutaric acid, itaconic acid, and mixtures thereof, in free or salified form.

20. A composition according to claim 3, characterized in that the modulating agent is chosen from propionic acid, citric acid, tartaric acid, lactic acid, malic acid, succinic acid, glutaric acid, itaconic acid, and mixtures thereof, in free or salified form.
